(19) United States
(12) Patent Application Publication
(10) Pub. No.: US 2015/0038885 A1
(43) Pub. Date: Feb. 5, 2015

(54) FOOT THERAPY APPARATUS

(71) Applicant: Zhigang ZHAO, Shenzhen (CN)

(72) Inventor: Zhigang ZHAO, Shenzhen (CN)

(21) Appl. No.: 14/519,105

(22) Filed: Oct. 20, 2014

Related U.S. Application Data
(63) Continuation-in-part of application No. PCT/CN2013/083529, filed on Sep. 16, 2013.

(30) Foreign Application Priority Data
Dec. 17, 2012 (CN) 201220698150.0

(51) Int. Cl. A61N 1/18; A63B 23/08

(52) U.S. Cl. CPC A61N 1/18; A63B 23/08
USPC 601/22

(57) ABSTRACT

A foot therapy apparatus, including a base, a pedal, an electric host, and an electrode. The electric host is disposed in the base and a display and/or an operation button of the electric host is disposed on the surface of the base. The pedal is connected to the base. The electrode is disposed at the upper surface of the pedal, and is connected to the electric host. The electric host is configured to drive the pedal to move, or to provide electric current for the electrode, or a combination thereof. The electric host includes a pedometer.

### CROSS-REFERENCE TO RELATED APPLICATIONS

This application is a continuation-in-part of International Patent Application No. PCT/CN2013/083529 with an international filing date of Sep. 16, 2013, designating the United States, now pending, and further claims priority benefits to Chinese Patent Application No. 201220698150.0 filed Dec. 17, 2012. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND OF THE INVENTION

1. Field of the Invention

The invention relates to a foot therapy apparatus.

2. Description of the Related Art

Existing foot therapy apparatus can only provide mechanical massage for feet, and the massage effect leaves much to be desired.

### SUMMARY OF THE INVENTION

In view of the above-described problems, it is one objective of the invention to provide a foot therapy apparatus that can provide both mechanical massage and electrostimulation.

To achieve the above objective, in accordance with one embodiment of the invention, there is provided a foot therapy apparatus, comprising a base, a pedal, an electric host, and an electrode. The electric host is disposed in the base, and a display and/or an operation button of the electric host is disposed on a surface of the base. The pedal is connected to the base. The electrode is disposed at an upper surface of the pedal, and is connected to the electric host; the electric host is configured to drive the pedal to move, or to provide electric current for the electrode, or a combination thereof. The electric host comprises a pedometer.

In a class of this embodiment, the electrode comprises a plurality of bulges which correspond to acupoints of feet.

In a class of this embodiment, the electrode is made from a conductive silicone or conductive metal.

In a class of this embodiment, the base comprises a pedal shaft, the pedal is connected to the pedal shaft, and the pedal is adapted to revolve around the pedal shaft or a pivot point.

In a class of this embodiment, the pedal shaft is disposed at two sides of the base, and the pedal is in rotary connection to the pedal shaft at two sides of the base.

In a class of this embodiment, the pedal is an integrated structure, and the electrode comprises two pieces disposed at two sides of the base.

In a class of this embodiment, the pedal comprises two pieces which rotate synchronously, or reversely rotate.

In a class of this embodiment, the pedal comprises a limit block for limiting the feet thereby preventing tumble or rotation at 360 degree; the pedal further comprises a buffer-braking structure.

In a class of this embodiment, a damper is disposed at a connection part between the pedal and the base, and the damper comprises a regulating knob.

In a class of this embodiment, the damper is a bidirectional spring.

Advantages according to embodiments of the invention are summarized as follows:

1. The rotary pedal is connected to the electric host, so that, on one hand, a user can exercise his/her own feet, on the other hand, the electrode can stimulate and treat the acupoints of the user, thereby improving the theraputic effect; and

2. The damper can regulate the rotational resistance of the pedal so that the user can control the massage strength as needed.

### BRIEF DESCRIPTION OF THE DRAWINGS

The invention is described hereinbelow with reference to the accompanying drawings, in which:

FIG. 1 is a schematic diagram of a foot therapy apparatus in accordance with one embodiment of the invention;

FIG. 2 is a left view of FIG. 1;

FIG. 3 is a bottom view of FIG. 1; and

FIG. 4 is a rear view of FIG. 1.

### DETAILED DESCRIPTION OF THE EMBODIMENTS

For further illustrating the invention, experiments detailing a foot therapy apparatus are described below. It should be noted that the following examples are intended to describe and not to limit the invention.

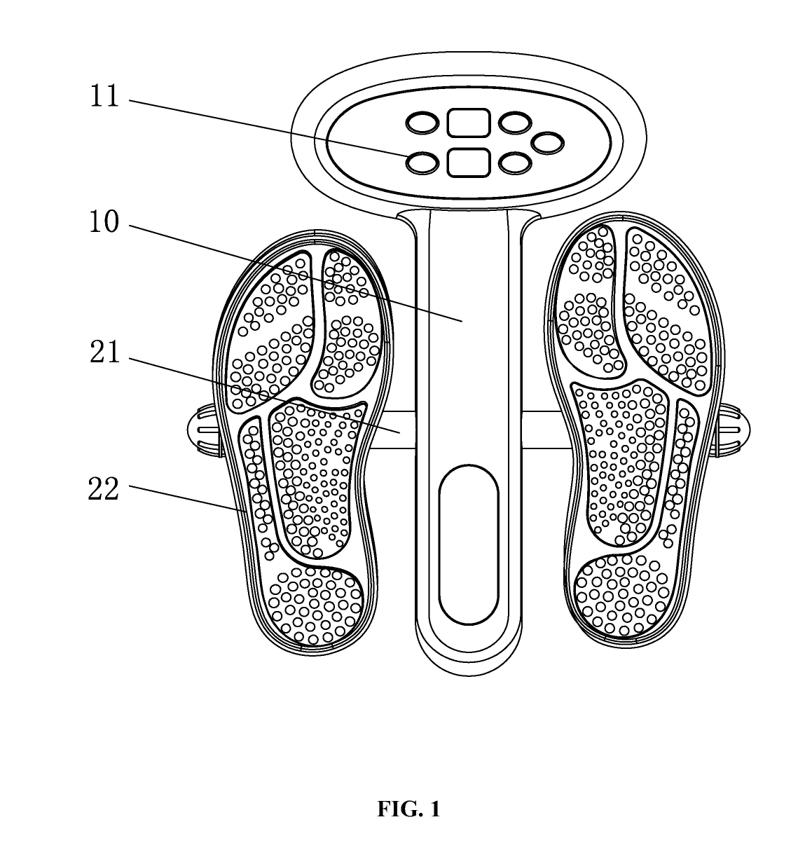

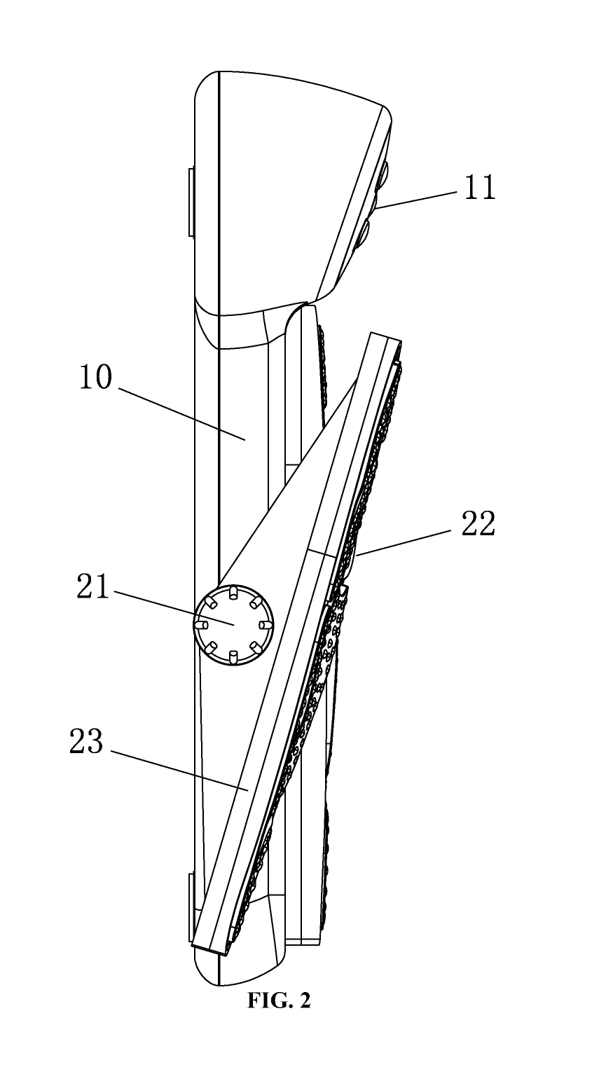

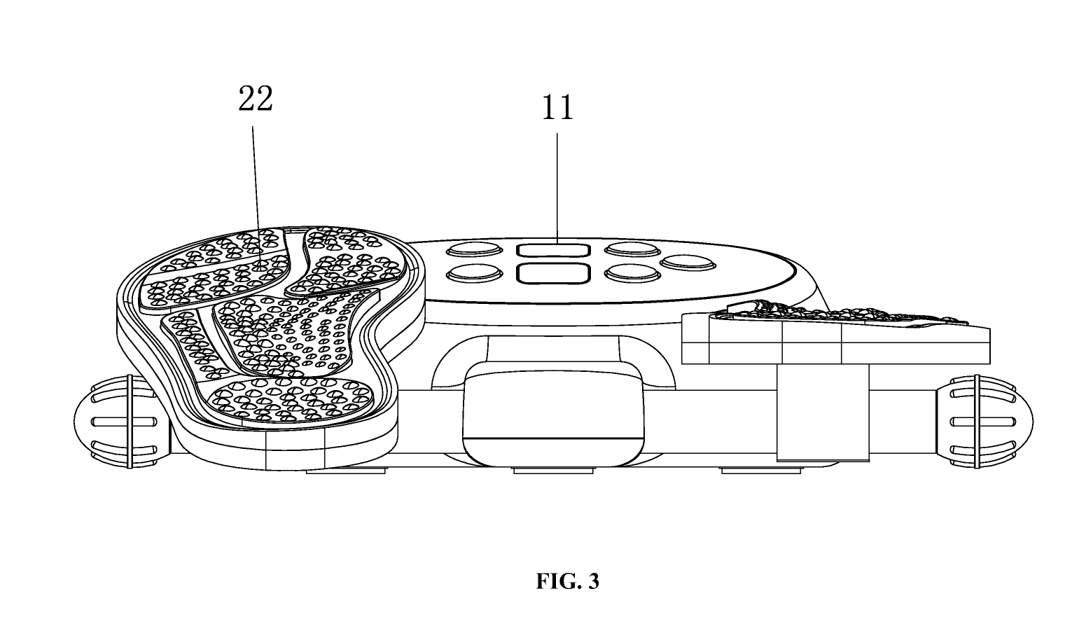

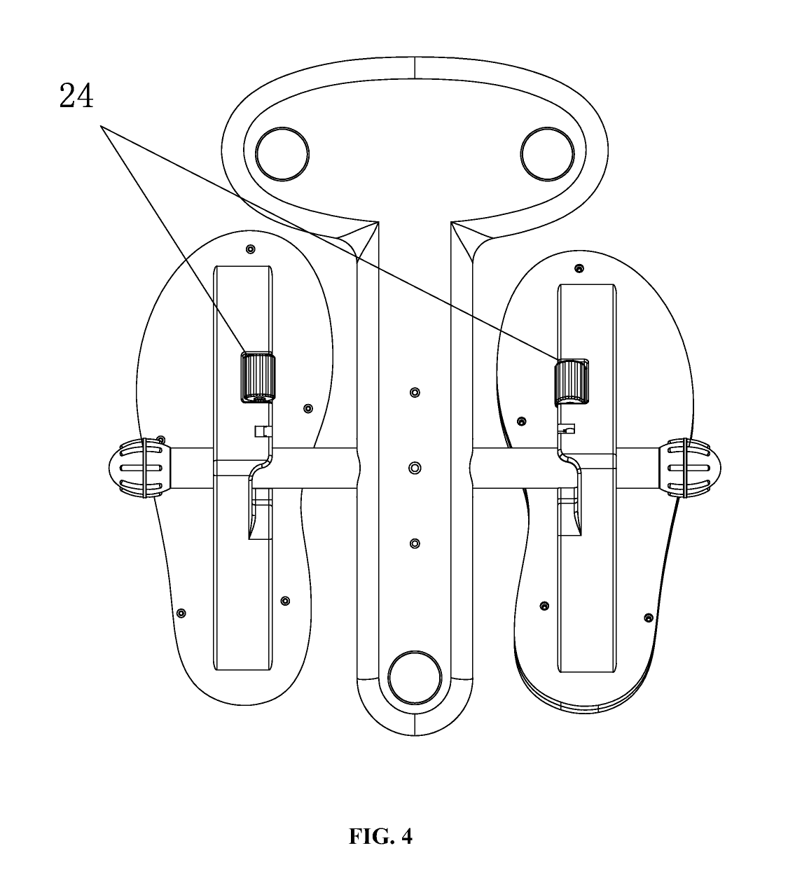

As shown in FIGS. 1-4, a foot therapy apparatus comprises a base 10, a pedal 23, an electric host (not shown in FIGS.), and an electrode 22.

The electric host is disposed in the base 10. An operation button 11 of the electric host is disposed on the surface of the base 10. Optionally, a display can be substituted for the operation button 11. As needed, the operation and the display can be both disposed on the base.

The pedal 23 is in a rotary connection to the base 10. In this example, the base 10 comprises a pedal shaft 21. The pedal 23 is in a rotary connection to the pedal shaft 21. Optionally, the pedal 23 is in a rotary connection to a part other than the pedal shaft 21 of the base 10. For example, the base 10 comprises an annular member, and the pedal 23 is in a rotary connection to the annular member of the base 10.

The electrode is made from conductive material such as conductive silicone or conductive metal. The electrode is in the shape of a plate, sheet, or dot. The bottom plate of the electrode 22 is covered on the upper surface of the pedal 23. The electrode 22 is in electric connection to the electric host and functions as an output terminal of the electric host.

In this example, the pedal shaft 21 is disposed at two sides of the base 10. The pedals 23 are in rotary connection to the pedal shaft 21 at two sides of the base 10. The pedals 23 can rotate synchronously, or alternately reversely rotate.

Optionally, the pedal 23 can be an integrated structure, and the electrode 22 comprises two pieces disposed on the pedal 23.

The pedal comprises a limit block for limiting the feet thereby preventing tumble or rotation at 360 degree. The pedal further comprises a buffer-braking structure.

To adjust the rotational resistance of the pedal 23 to meet the requirement for massage strength, a damper is disposed at the connection part between the pedal 23 and the base 10. The damper comprises a regulating knob 24. The damper is a bidirectional spring.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A foot therapy apparatus, comprising:
a) a base;
b) a pedal;
c) an electric host; and
d) an electrode;
wherein
the electric host is disposed in the base, and a display and/or an operation button of the electric host is disposed on a surface of the base;
the pedal is connected to the base;
the electrode is disposed at an upper surface of the pedal, and is connected to the electric host;
the electric host is configured to drive the pedal to move, or to provide electric current for the electrode, or a combination thereof; and
the electric host comprises a pedometer.

2. The foot therapy apparatus of claim 1, wherein the electrode comprises a plurality of bulges which correspond to acupoints of feet.

3. The foot therapy apparatus of claim 1, wherein the electrode is made from a conductive silicone or conductive metal.

4. The foot therapy apparatus of claim 1, wherein the base comprises a pedal shaft, the pedal is connected to the pedal shaft, and the pedal is adapted to revolve around the pedal shaft or a pivot point.

5. The foot therapy apparatus of claim 4, wherein the pedal shaft is disposed at two sides of the base, and the pedal is in rotary connection to the pedal shaft at two sides of the base.

6. The foot therapy apparatus of claim 4, wherein the pedal is an integrated structure, and the electrode comprises two pieces disposed at two sides of the base.

7. The foot therapy apparatus of claim 4, wherein the pedal comprises two pieces which rotate synchronously, or reversely rotate.

8. The foot therapy apparatus of claim 1, wherein the pedal comprises a limit block for limiting the feet thereby preventing tumble or rotation at 360 degree; the pedal further comprises a buffer-braking structure.

9. The foot therapy apparatus of claim 1, wherein a damper is disposed at a connection part between the pedal and the base, and the damper comprises a regulating knob.

10. The foot therapy apparatus of claim 1, wherein the damper is a bidirectional spring.
